(19) United States
(12) Patent Application Publication
(10) Pub. No.: US 2025/0381052 A1
(43) Pub. Date: Dec. 18, 2025

(54) USE OF BIOLOGICAL MATERIAL IN PREPARING PRODUCT FOR LIMITING gastric volume

(71) Applicants: Zhuoruan medical technologies (Suzhou) Co., Ltd, Suzhou (CN); Shanghai Zhuoruan medical technologies Co., Ltd, Shanghai (CN)

(72) Inventors: Yanyan WANG, Suzhou (CN); Wenyue CHENG, Suzhou (CN)

(73) Assignees: Zhuoruan medical technologies (Suzhou) Co., Ltd, Suzhou (CN); Shanghai Zhuoruan medical technologies Co., Ltd, Shanghai (CN)

(21) Appl. No.: 18/875,163

(22) PCT Filed: Jun. 16, 2023
(86) PCT No.: PCT/CN2023/100781

§ 371 Date: Dec. 16, 2024

(30) Foreign Application Priority Data
Jun. 17, 2022 (CN) 202210691945.7

(51) Int. Cl. A61F 5/00; A61L 27/36; A61L 27/56

(52) U.S. Cl. CPC A61F 5/0063; A61F 5/0076; A61F 5/0083; A61L 27/3604; A61L 27/362; A61L 27/3629; A61L 27/3683; A61L 27/56

(57) ABSTRACT

Provided is a use of a biological material in preparing a product for limiting gastric volume. The biological material is selected from a decellularized matrix, an artificial extracellular matrix and a high molecule compound. The wrapping of a remaining stomach after gastric volume reduction surgery by the biological material can effectively reduce the expansion of the gastric volume after the surgery for a long time. The gastric volume of the remaining stomach can be effectively maintained, avoiding the occurrence of weight regain caused by the expansion of the remaining stomach. The decellularized matrix and a biological material remolded tissue have the viscoelasticity equivalent to or close to natural tissue, avoiding gastric wall stiffness, and thus not affecting gastric wall peristalsis.

### FIELD OF TECHNOLOGY

The present disclosure relates to the field of biological materials, and in particular, to a use of a biological material in preparing a product for limiting gastric volume.

### BACKGROUND

Bariatric surgery has the highest long-term weight maintenance rate. The earliest bariatric surgery in China is gastric banding surgery, which uses a band to separate a small gastric pouch from a stomach and restrict the emptying place, to limit food reaching the lower digestive part from the upper part of the stomach. The gastric banding surgery is divided into vertical banded gastroplasty and adjustable gastric banding surgery, and the principle and process of the surgery are totally different from those of sleeve gastrectomy. The vertical band will seriously affect the life quality of patients. The adjustable band can adjust the tightness without modifying the structure such as the stomach and intestine, and the structure can be recovered after removing the band. However, when applied, it may cause gastric wall erosion, chronic gastric perforation, gastric obstruction, and even lead to patient death. Currently, this surgical method has been eliminated.

Currently, the mainstream surgical method in China is sleeve gastrectomy, which reduces the stomach capacity to limit food intake and reduce weight. The basic structure and function of the gastrointestinal tract remain unchanged after the surgery. The surgical method has the advantages of a good weight loss effect, simple operation, few complications, and no need to change the diet type after the surgery, and can be used as an independent bariatric surgery. The surgical method of sleeve gastrectomy is to fully dissociate the greater curvature of the stomach to the left diaphragmatic crus, and insert an esophageal dilator into the distal gastric antrum through the mouth. The esophageal dilator is used to standardize the size of the sleeve stomach. At a certain position away from the pylorus, the greater curvature side of the stomach is resected to form a new small tubular stomach with significantly reduced volume.

However, not all patients undergoing sleeve gastrectomy can maintain their weight after losing weight. Studies have reported that the incidence of weight regain two years after sleeve gastrectomy is 5.7%, and 6 years is 75.6%. The dilation of the sleeve stomach is one of the key causes of weight regain. Currently, the prevention and treatment methods are taking weight-loss drugs and reoperation, but weight-loss drugs such as orlistat and phentermine have obvious side effects and are not suitable for long-term use. Therefore, a method that can effectively restrict gastric dilation without gastric wall erosion, and does not affect gastric wall peristalsis has significant clinical value.

### SUMMARY

The present disclosure provides a use of a biological material in preparing a product for limiting gastric volume.

The biological material is selected from a decellularized matrix and an absorbable biological material.

The present disclosure provides a product for limiting gastric volume after gastric volume reduction surgery, which includes one or more biological materials selected from a decellularized matrix and an absorbable biological material.

In some embodiments, the absorbable biological material is an artificial extracellular matrix or a high molecule compound.

In some embodiments, the decellularized matrix includes a crosslinked or non-crosslinked acellular matrix, or a composite of crosslinked and non-crosslinked acellular matrix. The decellularized matrix is derived from one or more of a basement membrane of urinary bladder, a urinary bladder matrix, a small intestinal submucosa, a dermis, a pericardium, a pleura, a peritoneum, an intestinal mesentery, and a gastric submucosa.

In some embodiments, the product for limiting the gastric volume completely or partially wraps a circumference of a remaining stomach when applied, including major-circumference reinforcement, semi-circumference reinforcement and region reinforcement. In some embodiments, circumference reinforcement is in a range of above 20% of the circumference.

In some embodiments, gastric wall reinforcement with the product for limiting the gastric volume includes single-region reinforcement or multi-region reinforcement. In some embodiments, a reinforcement region has a width that effectively covers more than 20% of a length of a greater curvature of the stomach or a lesser curvature of the stomach.

In some embodiments, the biological material is provided with through holes.

In some embodiments, the biological material is one or a combination of sheet-like structure, strip-like structure and linear structure.

In some embodiments, the surface of the biological material is provided with an anti-adhesion structure.

In some embodiments, the biological material is provided with at least one notch. In some embodiments, the biological material is used for preparing the product for limiting the gastric volume after gastric volume reduction surgery.

In some embodiments, the gastric volume reduction surgery is sleeve gastrectomy or gastric bypass surgery.

The present disclosure further provides a method for limiting gastric volume after gastric volume reduction surgery, including:
(1) providing a product for limiting the gastric volume, which includes one or more biological materials;
(2) wrapping around the anterior and posterior gastric walls by using the biological material; and
(3) excising a part of a stomach tissue, while closing the biological material, so that the biological material circumferential wraps a formed remaining stomach.

In some embodiments, the gastric volume reduction surgery is sleeve gastrectomy, wherein the remaining stomach is a sleeve stomach, and the biological material wraps different positions of the sleeve stomach.

In some embodiments, the gastric volume reduction surgery is gastric bypass surgery, wherein the remaining stomach is a small gastric pouch, and the biological material wraps the small gastric pouch.

In some embodiments, the absorbable biological material is an artificial extracellular matrix or a high molecule compound; the decellularized matrix is a non-crosslinked material, a crosslinked material, or a composite of crosslinked and non-crosslinked acellular matrix. In some embodiments, the decellularized matrix is derived from one or more of a basement membrane of urinary bladder, a urinary bladder matrix, a small intestinal submucosa, a dermis, a pericardium, a pleura, a peritoneum, an intestinal mesentery, and a gastric submucosa.

In some embodiments, the biological material is provided with through holes. In some embodiments, the shape of the through holes is circular, square or rhombic. And in some embodiments, the through holes have a size of 0.1 to 20 mm.

In some embodiments, the through holes are distributed all over the biological material, or provided in a part of regions on the biological material. In some embodiments, the through holes are only provided in a middle section of the biological material.

In some embodiments, the biological material is one or a combination of sheet-like structure, strip-like structure and linear structure; and/or, the thickness of the biological material is not uniform.

In some embodiments, the biological material is provided with at least one notch.

In some embodiments, the surface of the biological material is provided with an anti-adhesion structure.

In some embodiments, the biological material completely or partially wraps a circumference of the remaining stomach. In some embodiments, the partial wrapping is selected from major-circumference reinforcement, semi-circumference reinforcement and region reinforcement. In some embodiments, circumference reinforcement is in a range of above 20% of the circumference.

In some embodiments, gastric wall reinforcement with the product for limiting the gastric volume is selected from single-region reinforcement and multi-region reinforcement. In some embodiments, a reinforcement region covers more than 20% of a length of a greater curvature of the stomach or a lesser curvature of the stomach. The present disclosure provides a use of a biological material in the preparation of the product for limiting the gastric volume, which has the following beneficial effects: the wrapping of a remaining stomach after gastric volume reduction surgery by the biological material can effectively reduce the expansion of the gastric volume after the surgery for a long time. The biological material has good mechanical performance in the early stage of implantation, which can limit the expansion of the stomach. And in the late stage of implantation, the fascia tissue remolded by the material forms a contraction device similar to an elastic band. On the one hand, the gastric volume of the remaining stomach can be effectively maintained, avoiding the occurrence of weight regain caused by the expansion of the remaining stomach. On the other hand, since the biological material and its remodeled tissues have certain elasticity of natural tissues, along with the peristalsis of the gastric wall, they will not cause the gastric wall to be stiff and avoid discomfort symptoms such as heartburn and nausea.

### BRIEF DESCRIPTION OF THE DRAWINGS

FIG. 1 is a schematic diagram of wrapping a remaining stomach by a biological material of the present disclosure, in which 1 is the biological material, 2 is a staple, and 3 is a suture.

FIG. 2 is a schematic diagram of the structure of a biological material of the present disclosure.

FIG. 3 is a schematic diagram of the structure of a biological material of the present disclosure.

FIG. 4 is a schematic diagram of the structure of a biological material of the present disclosure.

FIG. 5 is a schematic diagram of the structure of a biological material of the present disclosure.

FIG. 6 is a schematic diagram of the structure of a biological material of the present disclosure.

FIG. 7 is a schematic diagram of the structure of a biological material of the present disclosure.

FIG. 8 is a schematic diagram of the structure of a biological material of the present disclosure.

FIG. 9 is a schematic diagram of wrapping a remaining stomach by a biological material of the present disclosure.

FIG. 10 is a schematic diagram of wrapping a remaining stomach by a biological material of the present disclosure.

### DETAILED DESCRIPTION

Provided is a use of a biological material in preparing a product for limiting gastric volume, the biological material is selected from a decellularized matrix and an absorbable biological material.

The absorbable biological material is an artificial extracellular matrix or a high molecule compound.

The decellularized matrix is a new biological material that is obtained by treating allogeneic or xenogeneic tissues by decellularization, to remove the antigens that can cause immune rejection while completely retaining the ingredients and three-dimensional ultrastructure of extracellular matrix.

The decellularized matrix is selected from one or more of a basement membrane of urinary bladder, a urinary bladder matrix, a small intestinal submucosa, a dermis, a pericardium, a pleura, a peritoneum, an intestinal mesentery, and a gastric submucosa.

In some embodiments, the decellularized matrix is a material containing a complete basement membrane. The effectiveness of different biological materials varies. Multiple biological materials can be used simultaneously in a single product for limiting gastric volume, and in practical applications, appropriate selection and combination can be made according to the properties of different materials. For example, the wrap formed by porcine bladder basement membrane material is elastic and has small deformation. The porcine small intestinal submucosa material can better wrap part of the stomach body, but the stability of the overall wrapping is slightly poor. Dermis material and pericardium material have good shape stability but are slightly less integrated with the stomach body.

In some embodiments, the decellularized matrix is a non-crosslinked material, a crosslinked material, or a composite material of crosslinked and non-crosslinked.

Cross linking of the present disclosure refers to collagen molecules in a non-crosslinked membrane decellularized matrix that are bonded by covalent bonds within or between the molecules, or between the collagen molecules and other ingredients under the action of physical or chemical methods, to block the targets of collagenases, prolong the degradation time of the material after implantation, and stabilize the function of the repair regions, i.e., ensure that the reinforcement material can still ensure the reinforcement effect of the remaining stomach in various environments, including the surgery region complicated with infection.

The decellularized matrix is a composite material that can be commercially available, or obtained by a non-crosslinking method, a chemical crosslinking method, a physical crosslinking method, or a crosslinking combined with non-crosslinking method in the prior art.

The crosslinked decellularized matrix can have a sheet shape, a strip shape and a shape with large meshes.

The biological material is a multi-layer structure, and a part of the layers are crosslinked acellular matrices.

In some embodiments, the biological material is a 1-layer to 15-layer structure, for example.

The artificial extracellular matrix is a material that is prepared from decomposable natural or synthetic polymer materials and imitates the extracellular matrix within organisms. The material used to prepare the artificial extracellular matrix is, for example, collagen, etc.

The high molecule compound is, for example, one or more of a polymer or a copolymer from polyglycolic acid (PGA), polylactic acid-glycolic acid copolymer (PLGA), silk protein, fibrin, polyglycolic acid (PGA), polycaprolactone (PCL), polylactide, poly (p-dioxanone) (PDO), etc.

In some embodiments, the biological material is provided with through holes. The pore size, shape and distribution position of the through holes can be adjusted according to different functions. For example, the through holes have a diameter of 0.1 to 20 mm. In some embodiments, the diameter is 0.1 to 1.0 mm or 3 mm to 5 mm.

The through holes can be distributed all over the biological material or only provided in a part of the regions on the biological material, for example, only provided in the middle section of the biological material (as shown in FIGS. 2-6). There are no through holes provided at both ends of the biological material. Specifically, in some embodiments, no through holes are provided at 4-6 cm from both ends of the biological material.

The through holes can have a circular, square, rhombus, and trapezoid shape.

The through holes can reduce the influence of the shrinkage of the biological material on the gastric wall, and are beneficial to the outflow of the liquid on the surface of the gastric wall.

Dimensions such as the bending radian, shape, length and width of the biological material can be adjusted according to the size of a remaining stomach. The product for limiting gastric volume prepared from the biological material is attached to the surface of a remaining stomach smoothly, while maintaining a certain tension.

The biological material is one or a combination of sheet-like structure, strip-like structure and linear structure. In some embodiments, the biological material is a sheet-like structure, or a combined structure of sheet-like structure and strip-like structure and/or linear structure.

The biological material has a thickness of 0.05 to 2 mm. In some embodiments, the biological material has a thickness of 0.1 to 0.3 mm.

The biological material has a certain mechanical strength, and the tensile strength of the biological material is >0.5 N/cm. In some embodiments, the tensile strength of the biological material is >2 N/cm.

The biological material may have a structure with uneven thickness. In some embodiments, the central area and the anastomosis areas at both ends of the biological material are thin. The thin region of the central area is used to contact the neurovascular area of a lesser curvature of the stomach to reduce the influence on vagus nerve innervation; the thin regions at both ends are designed to reduce the influence of a linear cutting stapler on gastric wall disconnection and have a better anastomosis effect. The parts between the center and both ends, i.e., the parts located at the anterior and posterior gastric walls, have a relatively high thickness to better strengthen the gastric wall and limit the expansion of the gastric wall.

The biological material has a cross-sectional shape selected from a square shape, a circle shape, a trapezoid shape, a butterfly shape and other irregular shapes etc., and notches with various shapes such as a rounded corner, a sharp corner and a groove can also be provided on these shapes. These structures can better fit the gastric wall and bypass the main blood vessels in the stomach.

As shown in FIG. 2, the biological material has a rectangular cross-sectional shape with through holes. The length of A is 3 to 25 cm and the length of C is 2 to 10 cm.

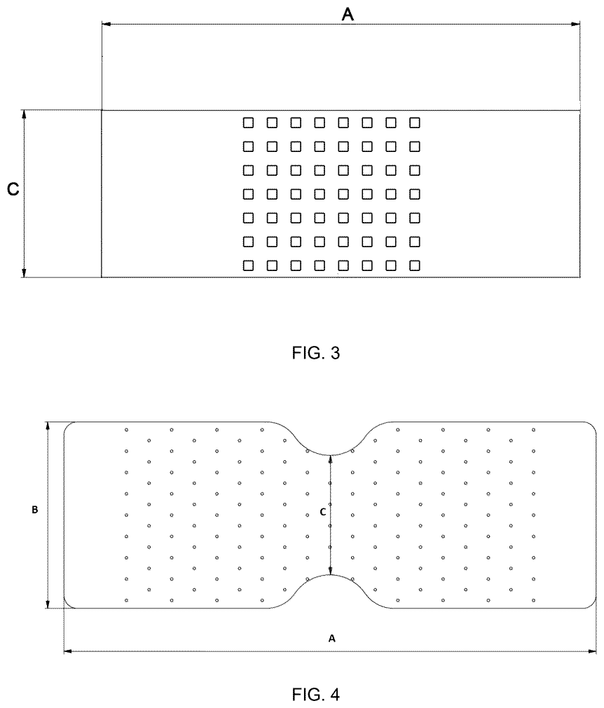

As shown in FIG. 3, the biological material has a rectangular cross-sectional shape with through holes. The through holes are distributed in the center of the material.

As shown in FIG. 4, the biological material has a rectangular cross-sectional shape, and rounded corners and grooves are added based on the rectangle. The grooves allow a better fit to the blood vessels in the stomach. The length of B is 5 to 15 cm, and the length of D is 2 to 10 cm.

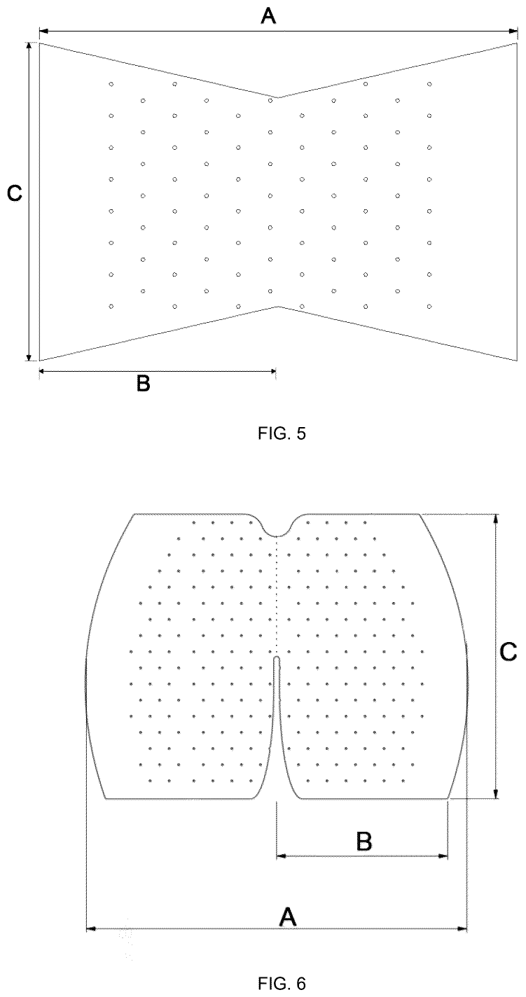

As shown in FIG. 5, the biological material has a butterfly cross-sectional shape with through holes. The length of A is 3 to 25 cm and the length of C is 2 to 10 cm. The butterfly design can achieve the same effective coverage ratio of reinforcement for a lesser curvature of the stomach and a greater curvature of the stomach.

As shown in FIG. 6, the biological material has a butterfly cross-sectional shape, and grooves and radians are added based on the butterfly shape, so that the residual gastric body can be wrapped more fit.

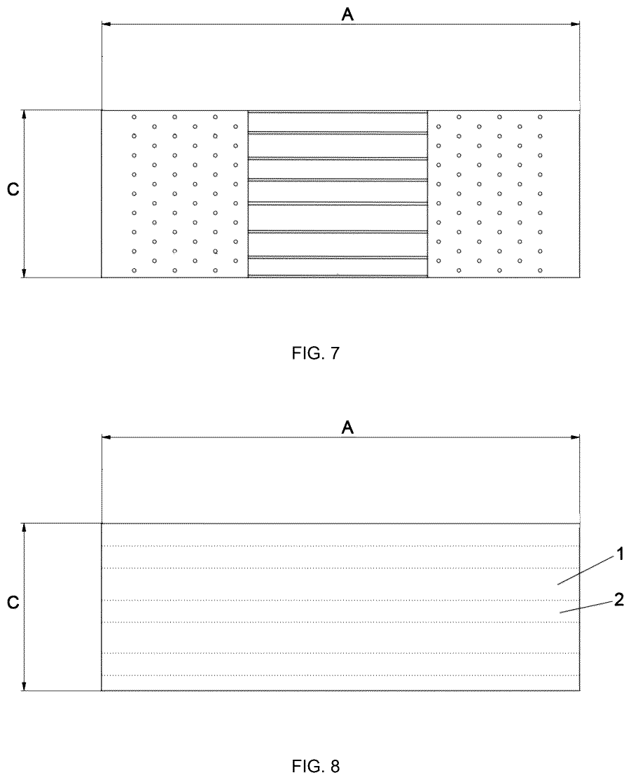

As shown in FIG. 7, the biological material has a shape of a combined structure of sheet-like structure and strip-like structure, to ensure the wrapping of a remaining stomach while reducing the influence on gastric peristalsis. The band has a width of 0.2 to 2 cm.

As shown in FIG. 8, the biological material comprises a sheet-like non-crosslinked material 1 and a strip-like crosslinked material 2. The crosslinked material can prolong the degradation time of the material and play a role in keeping the wrapping and binding of a remaining stomach even if the non-crosslinked material is degraded. The strip has a width of 0.2 to 2 cm.

The present disclosure provides a method for limiting gastric volume after gastric volume reduction surgery, the gastric volume reduction surgery is sleeve gastrectomy or gastric bypass surgery.

In one embodiment, the gastric volume reduction surgery is sleeve gastrectomy, which is implemented by laparoscopy or open surgery, and the method includes the following steps:

(1) After the abdominal cavity is entered and the greater omentum is completely dissociated, the omental bursa is entered, and dissociation upward along the greater curvature of the stomach is carried out continually to fully expose the fundus of the stomach, and separation is carried out to the esophagogastric junction at the left diaphragmatic crus.

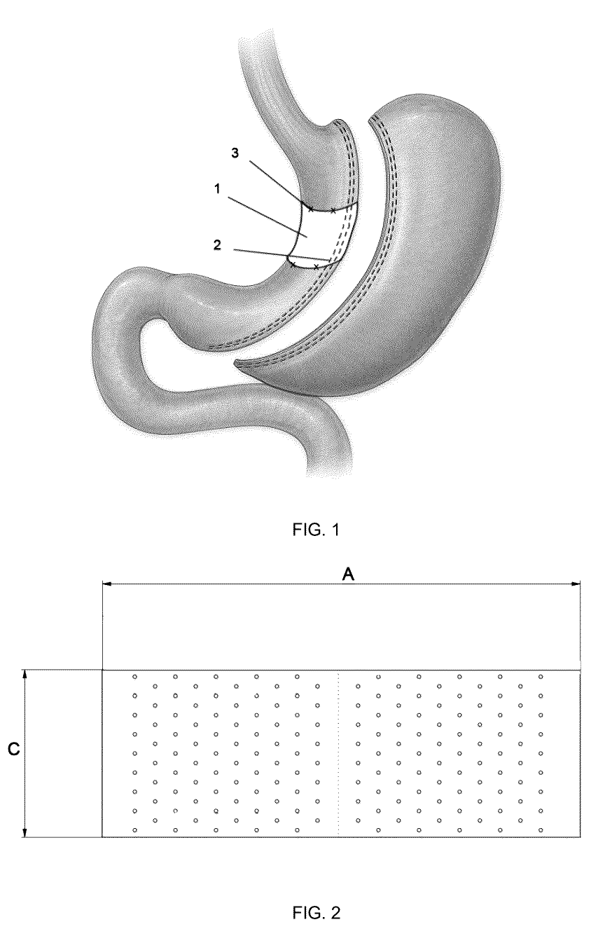

(2) A gastric tube is inserted through the mouth as support and passes through the pylorus and enters the duodenum as much as possible. The biological material is folded in half along the long axis with the folding line aligned with the lesser curvature of the stomach, allowing the anterior and posterior gastric walls to surround the biological material. A linear cutting stapler is used to cut the greater curvature of the stomach tissue along the supporting gastric tube from a certain position away from the pylorus while the biological material is closed, allowing the biological material to wrap the formed sleeve stomach around the circumference. As shown in FIGS. 1, 9, and 10, the biological material wraps the remaining stomach in different positions.

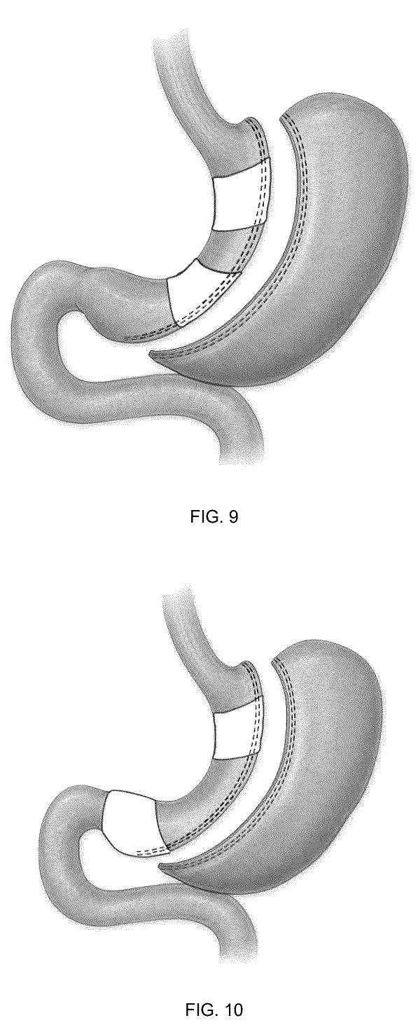

As shown in FIG. 1, the remaining stomach can be wrapped on the upper part. As shown in FIG. 9, the remaining stomach can be wrapped on the upper and lower parts simultaneously. As shown in FIG. 10, based on wrapping the remaining stomach on the upper part, the wrap of the gastric fundus is strengthened to prevent the gastric fundus from expanding, to further optimize the effect of limiting the gastric volume expansion. At the same time, to better fix the biological material, prevent the intestinal tube from drilling in and avoid anastomotic leakage, several intermittent and reinforced sutures can be selectively added.

In the case shown in FIGS. 1, 9, and 10, all the biological materials with the structures as shown in FIGS. 2, 3, 4, 5, 6, 7, and 8 can be used alone or in combination.

(3) If there are no signs of leakage observed, the gastric tube is removed and the abdominal cavity is closed.

In one embodiment, the gastric volume reduction surgery is gastric bypass surgery, and the method includes the following steps:

(1) Grasp the lesser curvature of the stomach, fold the biological material in half, wrap the biological material around the anterior and posterior gastric walls, use a linear cutting stapler to cut off the stomach on the lesser curvature side of the stomach, and close the biological material at the same time, so that the biological material forms a small stomach pouch wrapped circumferentially, and reserve the anastomosis between the intestine and the stomach. At the same time, to better fix the biological material, prevent the intestinal tube from drilling in, and avoid anastomotic leakage, several intermittent and reinforced sutures can be selectively added.

(2) The remaining operations are the same as those of conventional gastric bypass surgery. Refer to the following steps specifically: identify the Treitz ligament, cut off the jejunum at a certain distance from the Treitz ligament, anastomose the proximal stomach and the distal small intestine stump, and then anastomose the proximal jejunum stump and the distal small intestine at a certain distance from the anastomosis between the intestine and the stomach. The surface layer of the biological material is a dense structure to reduce the occurrence of tissue adhesion. In some embodiments, the surface of the biological material is provided with an anti-adhesion structure. The anti-adhesion structure is, for example, a coating or surface membrane with a dense structure.

The biological material is also subjected to hydration treatment. The hydration condition is soaking in normal saline for 3 to 10 min.

In some embodiments, the biological material is used for preparing a product for limiting gastric volume after gastric volume reduction surgery.

In some embodiments, the gastric volume reduction surgery is sleeve gastrectomy or gastric bypass surgery.

In some embodiments, the product for limiting the gastric volume after gastric volume reduction surgery is a stable contraction device for limiting the gastric volume.

The product for limiting the gastric volume includes one or more of the biological materials.

The product for limiting the gastric volume wraps the surface of a digestive tract, e.g., the surface of a stomach or the surface of each anastomosis, and forms strong tissues on the surface of a gastric wall to limit the expansion of the gastric wall, thereby reinforcing the gastric wall, limiting the gastric volume and reducing the probability of weight regain.

The method for fixing the product for limiting the gastric volume and the gastric wall is not limited, as long as the fixing method does not generate or form a centripetal force with low tension, does not cause gastric wall erosion and affect gastric wall peristalsis. The fixing method includes, for example, staple fix, suture fix, biological glue or degradable compound adhesion or other methods that can fix between a biological material and a gastric wall tissue.

The product for limiting the gastric volume can completely or partially wrap around circumference of a remaining stomach. The method for reinforcing a gastric wall by the product for limiting the gastric volume is selected from total-circumference wrapping, major-circumference reinforcement, semi-circumference reinforcement and region reinforcement. In some embodiments, circumference reinforcement is in a range of above 20% of the circumference.

In some embodiments, gastric wall reinforcement with the product for limiting the gastric volume includes single-region reinforcement or multi-region reinforcement.

The gastric wall reinforcement with the product for limiting the gastric volume has a width covering more than 20% of a length of a greater curvature of the stomach or a lesser curvature of the stomach.

The suture and the biological glue are located at any position of the ring formed by the biological material, and can be fixed with the gastric wall in intermittent points, to reduce the gap between the biological material and the remaining stomach, and prevent intestines from migrating into the gap between the biological material and the remaining stomach to cause intestinal obstruction and necrosis. It can be used independently to limit the gastric volume after gastric volume reduction surgery under the conditions of circumference reinforcement, semi-circumference reinforcement and region reinforcement.

The embodiments of the present disclosure are illustrated below through specific examples, and those skilled in the art can easily understand other advantages and effects of the present disclosure from the contents disclosed in this specification. The present disclosure can also be implemented or applied through other different detailed descriptions, and various details in this specification can be modified or changed based on different viewpoints and applications without departing from the spirit of the present disclosure.

Before further describing the detailed description of the present disclosure, it will be understood that the protection scope of the present disclosure is not limited to the following specific embodiments; also, it will be understood that the terms used in the embodiments of the present disclosure is for to describe specific embodiments, and not to limit the protection scope of the present disclosure. And in the specification and claims of the present disclosure, the singular forms “a”, “an” and “this” include the plural form unless the context clearly indicates otherwise.

When the embodiments give numerical ranges, it will be understood that unless otherwise specified in the present disclosure, two endpoints of each numerical range and any numerical value between the two endpoints can be selected. Unless otherwise defined, all technical and scientific terms used in the present disclosure have the same meaning as commonly understood by those skilled in the art. In addition to the specific methods, equipment and materials used in the embodiments, according to the mastery of the prior art by those skilled in the art and the records of the present disclosure, any methods, equipment and materials in the prior art similar to or equivalent to the methods, equipment and materials described in the embodiments of the present disclosure can be used to achieve the present disclosure.

### Embodiment 1 In Vitro Model

A fresh pig stomach was taken, and after sleeve gastrectomy, the volume was limited with the biological material as shown in FIG. 2. The range of circumference reinforcement was 0, 10, 20, 50 and 100%, and the wrapping length was 50% of the effective length of the lesser curvature of the stomach. The gastric body was completely removed from the esophagus to the duodenum, and the duodenum was tied tightly. A gastric tube connected with a manometer entered from the esophagus, and the esophagus and the gastric tube were tied tightly. The liquid was flushed from the manometer end until the pressure was 20 mmHg, and the circumference of the wrapping position was measured. After the pressure was increased continually to 40 mmHg, the circumference of the wrapping position was measured, with the results as follows:

| | | Circum- | Circum- | |
|---|---|---|---|---|
| | | ference at | ference at | Circum- |
| | 0 | 20 mmHg | 40 mmHg | ference |
| | pressure | pressure | pressure | growth rate |
| Surrounding | 9.42 | 14.5 | 17.0 | 80% |
| circumference | | | | |
| at 0% (cm) | | | | |
| Surrounding | 9.52 | 14.0 | 16.8 | 76% |
| circumference | | | | |
| at 10% (cm) | | | | |
| Surrounding | 9.55 | 11.7 | 11.9 | 24% |
| circumference | | | | |
| at 20% (cm) | | | | |
| Surrounding | 9.75 | 11.4 | 11.5 | 17% |
| circumference | | | | |
| at 50% (cm) | | | | |
| Surrounding | 9.90 | 11.0 | 11.2 | 13% |
| circumference | | | | |
| at 100% (cm) | | | | |

The above data show that the range of circumference reinforcement with the biological material of greater than 20% can significantly reduce gastric wall expansion and limit gastric volume.

### Embodiment 2 In Vivo Model

Large white pigs were selected as the experimental animal model. In the experimental group, different biological materials were used to wrap the gastric wall after gastric volume reduction surgery, and the experimental animals only undergoing gastric volume reduction surgery were used as the control group. The animals in each group were given the same amount of food every day without restricting their eating. Specific operation methods were as follows.

After the abdominal cavity was entered, the greater omentum was opened from outside the vascular arch in the middle of the greater curvature of the stomach, and resected in the direction of the gastric antrum, and then the right blood vessel of the omentum was ligated and severed at the root of the omentum, and then dissociated along the left side to the left blood vessel of the omentum, and ligated and severed. After the greater omentum was completely dissociated, the omental bursa was entered, and dissociation upward along the greater curvature of the stomach was carried out continually to fully expose the gastric fundus, and separation was carried out to the esophagogastric junction at the left diaphragmatic crus.

The mesentery of the lesser curvature of the stomach was completely separated from the posterior wall of the stomach, from the gastroduodenal artery to the esophagogastric junction at the right diaphragmatic crus, with the left gastric artery was reserved in the middle. The distance between the gastroduodenal artery and the left gastric artery was measured at the side of the lesser curvature of the stomach in its natural state. A supporting tube was inserted through the mouth into the duodenum along the side of the lesser curvature of the stomach. The biological material was aligned with the lesser curvature of the stomach, as shown in FIG. 1, allowing the anterior and posterior gastric walls to surround the biological material 1. After the biological material completely wrapped most of the distal stomach, the tissue of the greater curvature of the stomach was cut from the pyloric part, while the gastric wall tissue was cut at the side of the greater curvature of the stomach along the outside of the supporting tube with a linear stapler, and the edge of the biological material was fixed and closed by the staples 2, to wrap the remaining stomach. After the edge of the material was fixed, the area surrounded by the material was fixed in points with the suture 3 to prevent the intestinal canal from drilling in to form obstruction. In the same way, another piece of material was used to cover the gastric wall tissue, and the greater curvature of the stomach, the gastric wall tissue and the mesh were cut at the side of the greater curvature of the stomach along the outside of the supporting tube with a linear stapler, as shown in FIG. 9. The suture of the incised edge of the gastric fundus was strengthened and sutured with absorbable suture. The gastric tube was removed, and then the abdomen was closed.

The weight of the animals was recorded once a month after the surgery. The animals were sacrificed one year after the surgery. The gastric morphology, volume and adverse reactions of the remaining stomach were compared and observed. Circumference refers to the range where the material can cover the cross-section of the stomach based on the assumed cross-section of the stomach. Effective wrapping length refers to the wrapping length along the greater curvature of the stomach or the lesser curvature of the stomach.

TABLE 1
Grouping situation

| | | | | Effective |
|---|---|---|---|---|
| | | | | wrapping |
| | | | | length of |
| | | | | lesser |
| | | | | curvature |
| | Biological | | | of the |
| Group | material | Texture | Circumference | stomach |
| A | Rectangular, | porcine small intestinal | 100% | 80% |
| | without through | submucosa/porcine bladder | | |
| | holes | basement membrane composite | | |
| | | decellularized matrix | | |
| B | Rectangular, | porcine small intestinal | 100% | 50% |
| | with through | submucosa/porcine bladder | | |
| | holes | basement membrane composite | | |
| | | decellularized matrix | | |
| C | Butterfly- | porcine small intestinal | 100% | 20% |
| | shaped, without | submucosa/porcine bladder | | |
| | through holes | basement membrane composite | | |
| | | decellularized matrix | | |
| D | Rectangular, | PGA membrane | 50% | 20% |
| | without through | | | |
| | holes | | | |
| E | Butterfly- | porcine small intestinal | 20% | 20% |
| | shaped, with | submucosa/porcine bladder | | |
| | through holes | basement membrane composite | | |
| | | decellularized matrix | | |
| F | Butterfly- | porcine small intestinal | 10% | 10% |
| | shaped, with | submucosa/porcine bladder | | |
| | through holes | basement membrane composite | | |
| | | decellularized matrix | | |
| G | null | null | null | null |

TABLE 2
Changes of residual gastric volume and
body weight one year after surgery

| | The change ratio of residual | The change ratio of body |
|---|---|---|
| | gastric volume one year after | weight one year after |
| | surgery compared with that | surgery compared with that |
| Group | immediately after surgery | immediately after surgery |
| A | 17% | −30% |
| B | 13% | −34% |
| C | 25% | −15% |
| D | 35% | −12% |
| E | 40% | −11% |
| F | 103% | +56% |
| G | 207% | +97% |

The above examples are intended to illustrate the disclosed embodiments of the present disclosure, and should not be the limitation of the present disclosure. In addition, various modifications listed herein and changes in methods in the present disclosure are obvious to those skilled in the art without departing from the scope and spirit of the present disclosure. Although the present disclosure has been described in detail concerning various specific embodiments, it should be understood that the present disclosure should not be limited to these specific embodiments. In fact, various modifications as mentioned above that are obvious to those skilled in the art to obtain the present disclosure should be included in the scope of the present disclosure.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method for limiting stomach volume, comprising wrapping anterior and posterior walls of a stomach with a biological material;
wherein the biological material is a decellularized matrix, and has a non-uniform thickness ranging from 0.05 to 2 mm; wherein the biological material is provided with through holes with a diameter of 0.1 to 20 mm;
wherein the biological material is configured to wrap a surface of the stomach, to reinforce a gastric wall by completely or partially wrapping a circumference of the residual stomach, wherein a reinforcement is in a range of above 20% of the circumference of the residual stomach.

22. The method of claim 21, wherein the decellularized matrix is a non-crosslinked material, a crosslinked material, or a crosslinked-non-crosslinked composite material.

23. The method of claim 22, wherein the decellularized matrix is derived from one or more of a bladder basement membrane, small intestinal submucosa, dermis, pericardium, pleura, peritoneum, mesentery, and gastric submucosa.

24. The method of claim 21, wherein the reinforcement of the gastric wall by biological material is single-region reinforcement or multi-region reinforcement.

25. The method of claim 24, wherein a reinforcement region covers more than 20% of a length of a greater curvature or a lesser curvature of the stomach.

26. The method of claim 21, wherein the shape of the through hole is circular, square, or rhombic.

27. The method of claim 21, wherein the through holes are distributed all over the biological material, or provided in a part of regions on the biological material.

28. The method of claim 27, wherein the through holes are only provided in a middle section of the biological material.

29. The method of claim 21, wherein the biological material is one or a combination of sheet-like structure, strip-like structure, and linear structure.

30. The method of claim 21, wherein the biological material is provided with one or more notches.

31. The method of claim 21, wherein the surface of the biological material is provided with an anti-adhesion structure.

32. The method of claim 21, wherein the biological material is used for preparing a product for limiting the stomach volume after gastric volume reduction surgery.

33. The method of claim 32, wherein the gastric volume reduction surgery is sleeve gastrectomy or gastric bypass surgery.
